(19) United States
(12) Patent Application Publication
(10) Pub. No.: US 2020/0000686 A1
(43) Pub. Date: Jan. 2, 2020

(54) DEODORANT STICKS

(71) Applicant: The Procter & Gamble Company, Cincinnati, OH (US)

(72) Inventor: David Frederick Swaile, Cincinnati, OH (US)

(21) Appl. No.: 16/450,159

(22) Filed: Jun. 24, 2019

Related U.S. Application Data
(60) Provisional application No. 62/691,315, filed on Jun. 28, 2018.

(51) Int. Cl. A61K 8/02; A61K 8/34; A61Q 15/00; A61K 8/37

(52) U.S. Cl. CPC A61K 8/0229; A61K 8/345; A61Q 15/00; A61K 2800/77; A61K 2800/30; A61K 2800/42; A61K 8/37

(57) ABSTRACT

A deodorant composition comprising from about 25% to about 70%, by weight of the composition, of an emollient selected from the group consisting of a propoxylated fatty alcohol, a propoxylated fatty acid, an ethoxylated propoxylated fatty alcohol, an ethoxylate propoxylated fatty acid and combinations thereof, each emollient having a molecular weight of at least about 750 Daltons; and from about 2% to about 10% of a fatty acid salt gellant.

### CROSS REFERENCE TO RELATED APPLICATION

This application claims the benefit of U.S. Provisional Application No. 62/691,315, filed Jun. 28, 2018, the substance of which is incorporated herein by reference.

### FIELD

Deodorant compositions comprising a high molecular weight water-dispersible emollient and a gellating agent.

### BACKGROUND

Most deodorant sticks are composed of small polyhydric alcohols (i.e. propylene glycol and dipropylene glycol), water, and a fatty acid salt thickener. Also, they often have a fragrance and an antibacterial to provide odor protection by masking malodor and/or controlling odor causing bacterial. Such a formulation design creates a lubricous feel at application, no visible white residue on skin or clothes, and a clean feel throughout the day that many consumers desire. Unfortunately, this product design can also result in much of the product penetrating the skin, including the fragrance or antibacterial, thereby removing the ability of those materials to provide the desired benefit. Attempts at creating new deodorant formulations typically comprise a majority of emollients with molecular weights of less than 250 Daltons that are either volatile or capable of rapidly penetrating the skin.

Therefore, there is a need for a deodorant stick that can provide the desirable aesthetic benefits consumers expect, while reducing the penetration into the skin.

### SUMMARY

A deodorant stick comprising from about 25% to about 70%, by weight of the composition, of an emollient selected from the group consisting of a propoxylated fatty alcohol, a propoxylated fatty acid, an ethoxylated propoxylated fatty alcohol, an ethoxylate propoxylated fatty acid and combinations thereof, each emollient having a molecular weight of at least about 750 Daltons; and from about 2% to about 10% of a fatty acid salt gellant.

### DETAILED DESCRIPTION

The components and/or steps, including those which may optionally be added, of the various embodiments of the present invention, are described in detail below.

All documents cited are, in relevant part, incorporated herein by reference; the citation of any document is not to be construed as an admission that it is prior art with respect to the present invention.

All ratios are weight ratios unless specifically stated otherwise.

All temperatures are in degrees Celsius, unless specifically stated otherwise.

Except as otherwise noted, all amounts including quantities, percentages, portions, and proportions, are understood to be modified by the word “about”, and amounts are not intended to indicate significant digits.

Except as otherwise noted, the articles “a”, “an”, and “the” mean “one or more”.

Herein, “comprising” means that other steps and other ingredients which do not affect the end result can be added. This term encompasses the terms “consisting of” and “consisting essentially of”. The compositions and methods/processes of the present invention can comprise, consist of, and consist essentially of the essential elements and limitations of the invention described herein, as well as any of the additional or optional ingredients, components, steps, or limitations described herein.

Herein, “effective” means an amount of a subject active high enough to provide a significant positive modification of the condition to be treated. An effective amount of the subject active will vary with the particular condition being treated, the severity of the condition, the duration of the treatment, the nature of concurrent treatment, and like factors.

The term “ambient conditions” as used herein refers to surrounding conditions under about one atmosphere of pressure, at about 50% relative humidity, and at about 25° C., unless otherwise specified. All values, amounts, and measurements described herein are obtained under ambient conditions unless otherwise specified.

The term “polarity” as used herein is defined by the Hansen Solubility Parameter for solubility.

“Substantially free of” refers to about 2% or less, about 1% or less, or about 0.1% or less of a stated ingredient. “Free of” refers to no detectable amount of the stated ingredient or thing.

The term “volatile” as used herein refers to those materials that have a measurable vapor pressure at 25° C. Such vapor pressures typically range from about 0.01 millimeters of Mercury (mm Hg) to about 6 mmHg, more typically from about 0.02 mmHg to about 1.5 mmHg; and have an average boiling point at one (1) atmosphere of pressure of less than about 250° C., more typically less than about 235° C. Conversely, the term “non-volatile” refers to those materials that are not “volatile” as defined herein.

“Deodorant composition” as used herein refers to a composition that is applied to at least a portion of the body, which is used to combat body odor.

“Leave-on” as used herein refers to a composition that is designed to be applied to at least a portion of the body and then left on that portion of the body.

### I. High Molecular Weight Liquid Emollient

The deodorant compositions of the present invention may comprise one or more emollients with a high molecular weight. The present inventors have discovered that the high molecular weights and corresponding larger sizes of such materials result in the materials and the overall composition remaining on the surface of the skin, rather than penetrating into the skin. This allows the benefits of the deodorant, such as those delivered by the fragrances or antibacterials, to increase and/or last longer.

In some cases, the high molecular weight emollients may have a molecular weight of at least about 750 Daltons, in some other cases, at least about 1000 Daltons, and in some other cases, at least about 1500 Daltons. The emollients used may be liquid. Suitable high molecular weight or liquid emollients may include, but are not limited to, propoxylated fatty alcohols, propoxylated fatty acids, ethoxylated propoxylated fatty alcohols, ethoxylate propoxylated fatty acids, and combinations thereof. Suitable high molecular weight or liquid emollients may include propoxylated fatty acids and propoxylated fatty alcohols, such as PPG-15 stearyl ether, PPG-11 Stearyl ether, PPG-15 Lauryl ether, PPG-11 Lauryl ether, PPG-15 myristyl ether, PPG-11 myristyl ether, PPG-14 butyl ether, and PPG-30 Cetyl ether. As used herein, fatty alcohol or fatty acid chains of the high molecular weight emollients include linear or branched alkyl chains with more than 4 carbon atoms. Typical chain lengths are from 4 to 28 atoms, with some embodiments having chain lengths of 4 to 18 carbon atoms.

The deodorant compositions of the present invention may comprise at least about 25% of one or more high molecular weight or liquid emollients, in some embodiments at least about 30%, at least about 35%, or at least about 40%, by weight of the composition. In some embodiments, the deodorant composition may comprise from about 25% to about 50% of one or more high molecular weight or liquid emollients, or in some embodiments from about 25% to about 70%, by weight of the composition. In some embodiments, the deodorant composition may comprise from about 25% to about 70% of an propoxylated fatty alcohols, propoxylated fatty acids, ethoxylated propoxylated fatty alcohols, ethoxylate propoxylated fatty acids, or combinations thereof, that have a molecular weight of at least about 750 Daltons. In some embodiments, the deodorant composition may comprise from about 30% to about 50%, from about 35% to about 50%, from about 40% to about 50%, from about 30% to about 70%, from about 40% to about 70%, from about 40% to about 60%, or from about 50% to about 70%, by weight of the composition, of one or more emollients having a molecular weight of at least about 750 Daltons, or of liquid emollients.

In some embodiments, the emollient may be water-dispersible, meaning that while not soluble in water, the emollient easily forms small to minute particles when mixed with water. Being water-disperable allows these material to be easily removed from skin during washing. Moreover in some embodiments, the emollient will have a viscosity of less then 500 cps, less then 200 cps or less than 100 cps. This viscosity range is capable of providing a light feel on skin which is desirable by some consumers.

Along with at least a high molecular weight emollient, the deodorant composition may comprise a fatty acid salt gellant or thickener. Fatty acid salt gellants are capable of creating the desired solid structure at relatively low concentrations (10% or less of the formula). Moreover, these gellants sometimes form twisted fiber crystal structures that allow some embodiments of the instant invention to be translucent or transparent. The deodorant composition may comprise from about 2% to about 10% of the fatty acid salt gellant, in some embodiments, from about 3% to about 6%, and in some embodiments, about 5% of the fatty acid salt gellant, by weight of the composition.

### Additional Emollients

The deodorant compositions of the present invention may also comprise additional emollients with molecular weights below 750 Daltons to provide a desired feel, to solubilize deodorant actives or fragrances, and to enable solubilization of the fatty acid gellant during product making. One particular type of additional emollient are polyhydric alcohols, which are typically added at a level of at most about 30%. Suitable polyhydric alcohols may include, but are not limited to, propylene glycol, dipropylene glycol, tripropylene glycol, low molecular weight polypropylene glycols, ethylene glycol, diethylene glycol, triethylene glycol, PEG-4, PEG-8, 1, 2 pentanediol, 1,2 hexanediol, hexylene glycol, trimethylene glycol, glycerine, sorbitol, and combinations thereof. The deodorant compositions may comprise the additional polyhydric alcohol emollients cumulatively at most about 30% by weight of the composition. In some embodiments, the deodorant compositions may comprise the polyhydric alcohol emollients cumulatively from about 5% to about 30%, from about 10% to about 27%, or from about 15% to about 25%, by weight of the composition.

Other additional emollients may include C2 to C20 monohydric alcohols, C2 to C20 dyhydric or polyhydric alcohols, alkyl ethers of polyhydric and monohydric alcohols, volatile silicone emollients such as cyclopentasiloxane, non-volatile silicone emollients such as dimethicone, mineral oils, polydecenes, petrolatum, and combinations thereof. Further examples of suitable additional emollients may include isopropyl myristate, butyl stearate, cetyl octanoate, butyl myristate, myristyl myristate, C12-15 alkylbenzoate (e.g., Finsolv™), octyldodecanol, isostearyl isostearate, octododecyl benzoate, isostearyl lactate, isostearyl palmitate, isobutyl stearate, dimethicone, and any mixtures thereof.

The deodorant compositions of the present invention may be formulated as an aqueous or anhydrous composition. In some embodiments that are aqueous, the composition may comprise from about 2% to about 8% water, by weight of the composition, in some embodiments from about 3% to about 5% water, and in some embodiments about 4% to about 5% water, by weight of the composition.

The deodorant compositions of the present invention may have a hardness measured by a penetration value of at most about 120 units, as determined by the test method detailed below. In some embodiments, the hardness may be from about 80 units to about 120 units.

### II. Other Deodorant Components

The deodorant composition may also include additional ingredients like, for example, solubilizers, chelants, anti-oxidants, fragrances, encapsulates, powders, structurants, thickeners, gelling agents, deodorant actives, other actives, preservatives, dyes, and combinations thereof, etc.

### Deodorant Actives

Suitable optional deodorant actives may include any topical material that is known or otherwise effective in preventing or eliminating malodor associated with perspiration. Suitable deodorant actives may be selected from the group consisting of antibacterial agents (e.g., bacteriocides, fungicides), malodor-absorbing material, and combinations thereof. For example, antibacterial agents may comprise cetyl-trimethylammonium bromide, cetyl pyridinium chloride, benzethonium chloride, diisobutyl phenoxy ethoxy ethyl dimethyl benzyl ammonium chloride, sodium N-lauryl sarcosine, sodium N-palmethyl sarcosine, lauroyl sarcosine, N-myristoyl glycine, potassium N-lauryl sarcosine, trimethyl ammonium chloride, sodium aluminum chlorohydroxy lactate, triethyl citrate, tricetylmethyl ammonium chloride, 2,4,4′-trichloro-2′-hydroxy diphenyl ether (triclosan), 3,4,4′-trichlorocarbanilide (triclocarban), diaminoalkyl amides such as L-lysine hexadecyl amide, heavy metal salts of citrate, salicylate, and piroctose, especially zinc salts, and acids thereof, heavy metal salts of pyrithione, especially zinc pyrithione, zinc phenolsulfate, farnesol, and combinations thereof. The concentration of the optional deodorant active may range from about 0.001%, from about 0.01%, of from about 0.1%, by weight of the composition to about 20%, to about 10%, to about 5%, or to about 1%, by weight of the composition.

Some embodiments may be aluminum-free, or substantially free of aluminum. In some embodiments, antibacterials may be selected from the group consisting of 2-Pyridinol-N-oxide (piroctone olamine), lupamin, beryllium carbonate, magnesium carbonate, calcium carbonate, magnesium hydroxide, magnesium hydroxide and magnesium carbonate hydroxide, partially carbonated magnesium hydroxide, potassium carbonate, potassium bicarbonate, sodium carbonate, sodium sesquicarbonate, baking soda, hexamidine, zinc carbonate, thymol, polyvinyl formate, salycilic acid, niacinamide and combinations thereof.

### Additional Structurants

The deodorant compositions may also comprise one or more addition structurants to provide the solid stick deodorant composition with the desired viscosity, rheology, texture and/or hardness, or to otherwise help suspend any dispersed solids or liquids within the composition. The one or more structurants may comprise at least one wax. The term “structurant” may also include any hydrophobic material known or otherwise effective in providing suspending, gelling, viscosifying, solidifying, or thickening properties to the composition or which otherwise provide structure to the solid stick deodorant composition. These structurants may include, for example, gelling agents, polymeric or nonpolymeric agents, inorganic thickening agents, or viscosifying agents. The thickening agents may include, for example, organic solids, silicone solids, crystalline or other gellants, inorganic particulates such as clays or silicas, or combinations thereof.

Waxes may be natural or synthetic materials. In some instances, one or more (or all) of the waxes present in the solid stick deodorant composition may have a melt temperature less than about 90° C. 85° C., 80° C. 75° C., 70° C. or 60° C. Some examples include natural vegetable waxes such as, for example, candelilla wax, carnauba wax, Japan wax, espartograss wax, cork wax, guaruma wax, rice oil wax, sugar cane wax, ouricury wax, montan wax, sunflower wax, fruit waxes, such as orange waxes, lemon waxes, grapefruit wax, bayberry wax, and animal waxes such as, for example, beeswax, shellac wax, spermaceti, wool wax and uropygial fat. Natural waxes may include the mineral waxes, such as ceresine and ozocerite for example, or the petrochemical waxes, for example petrolatum, paraffin waxes and microwaxes. Chemically modified waxes may be used, such as, for example, montan ester waxes, sasol waxes and hydrogenated jojoba waxes, Synthetic waxes include, for example, a polyethylene, a polymethylene, or a combination thereof.

The wax may also be selected from the group of esters of saturated and/or unsaturated, branched and/or unbranched alkanecarboxylic acids and saturated and/or unsaturated, branched and/or unbranched alcohols, from the group of esters of aromatic carboxylic acids, dicarboxylic acids, tricarboxylic acids and hydroxycarboxylic acids (for example 12-hydroxystearic acid) and saturated and/or unsaturated, branched and/or unbranched alcohols and also from the group of lactides of long-chain hydroxycarboxylic acids. Wax components such as these include, for example, C16-40 alkyl stearates, C20-40 alkyl stearates (for example Kesterwachs (Registered trademark K82H), C20-40 dialkyl esters of dimer acids, C18-38 alkyl hydroxystearoyl stearates or C20-40 alkyl erucates. Other suitable waxes which may be used include C30-50 alkyl beeswax, tristearyl citrate, triisostearyl citrate, stearyl heptanoate, stearyl octanoate, trilauryl citrate, ethylene glycol dipalmitate, ethylene glycol distearate, ethylene glycol di(12-hydroxystearate), stearyl stearate, palmityl stearate, stearyl behenate, cetyl ester, cetearyl behenate and behenyl behenate. Silicone waxes may also be used.

Some preferred examples of acceptable non-polar waxes include glyceryl tribehenate, polyethylene, polymethylene (e.g., Accumelt 68 and 78 available from International Group, Inc., USA), C₁₈-C₃₆ triglyceride (e.g., Synchrowax HGL-C available from Croda, Inc., USA), hydrogenated high euricic aid rapeseed oil (hear stearine), ozokerite and combinations thereof. Some preferred examples of acceptable polar waxes include stearyl alcohol, hydrogenated castor oil, myristyl alcohol, cetyl alcohol, and combinations thereof. The wax may comprise a blend of polar and non-polar waxes. For example, a combination of polar and non-polar waxes may be selected from the list above. In some instances, the wax may have a melt point above 65° C., more typically from about 65° C. to about 130° C. Some suitable polymethylenes may have a melting point from about 65° C. to about 75° C. Examples of suitable polyethylenes include those with a melting point from about 60° C. to about 95° C. Other high melting point waxes are described in U.S. Pat. No. 4,049,792, Elsnau, issued Sep. 20, 1977. Solid stick deodorant compositions may have a total wax concentration from about 10%, 12%, or 14% to about 25%, 20%, 18% or 16% by weight of the composition.

The compositions may also comprise one or more structurants other than wax. For example, one or more gelling agents may be included. Some non-limiting examples of suitable gelling agents include fatty acid gellants, salts of fatty acids, hydroxyl acids, hydroxyl acid gellants, esters and amides of fatty acid or hydroxyl fatty acid gellants, cholesterolic materials, dibenzylidene alditols, lanolinolic materials, fatty alcohols, triglycerides, sucrose esters such as SEFA behenate, inorganic materials such as clays or silicas, other amide or polyamide gellants, and mixtures thereof.

Suitable gelling agents include fatty acid gellants such as fatty acid and hydroxyl or alpha hydroxyl fatty acids, having from about 10 to about 40 carbon atoms, and ester and amides of such gelling agents. Non-limiting examples of such gelling agents include, but are not limited to, 12-hydroxystearic acid, 12-hydroxylauric acid, 16-hydroxyhexadecanoic acid, behenic acid, eurcic acid, stearic acid, caprylic acid, lauric acid, isostearic acid, and combinations thereof. Preferred gelling agents are 12-hydroxystearic acid, esters of 12-hydroxystearic acid, amides of 12-hydroxystearic acid and combinations thereof.

Other suitable gelling agents include amide gellants such as di-substituted or branched monoamide gellants, monsubstituted or branched diamide gellants, triamide gellants, and combinations thereof, including n-acyl amino acid derivatives such as n-acyl amino acid amides, n-acyl amino acid esters prepared from glutamic acid, lysine, glutamine, aspartic acid, and combinations thereof. Other suitable amide gelling agents are described in U.S. Pat. No. 5,429,816, issued Jul. 4, 1995, and U.S. Pat. No. 5,840,287, filed Dec. 20, 1996.

Still other examples of suitable gelling agents include fatty alcohols having at least about 8 carbon atoms, at least about 12 carbon atoms but no more than about 40 carbon atoms, no more than about 30 carbon atoms, or no more than about 18 carbon atoms. For example, fatty alcohols include but are not limited to cetyl alcohol, myristyl alcohol, stearyl alcohol and combinations thereof.

Non limiting examples of suitable tryiglyceride gellants include tristearin, hydrogenated vegetable oil, trihydroxysterin (Thixcin® R, available from Rheox, Inc.), rape seed oil, castor wax, fish oils, tripalmitin, Syncrowax® HRC and Syncrowax® HGL-C(Syncrowax® available from Croda, Inc.).

Some other structurants for use in the solid stick compositions may include inorganic particulate thickening agents such as clays and colloidal pyrogenic silica pigments. For example, colloidal pyrogenic silica pigments such as Cab-O-Sil®, a submicroscopic particulated pyrogenic silica may be used. Other known or otherwise effective inorganic particulate thickening agents that are commonly used in the art can also be used in the solid compositions of the present invention. Concentrations of particulate thickening agents may range, for example, from about 0.1%, about 1%, or about 5%; to about 35%, about 15%, about 10% or about 8%, by weight of the composition.

Suitable clay structurants include montmorillonite clays, examples of which include bentonites, hectorites, and colloidal magnesium aluminum silicates. These and other suitable clays may be hydrophobically treated, and when so treated will generally be used in combination with a clay activator. Non-limiting examples of suitable clay activators include propylene carbonate, ethanol, and combinations thereof. When clay activators are present, the amount of clay activator will typically range from about 40%, about 25%, or about 15%; to about 75%, about 60%, or about 50%, by weight of the clay.

A solid stick composition may contain from about 15% to about 25%, by weight of the composition, of structurants.

### Perfumes and Fragrance Delivery

The compositions herein may include microcapsules. The microcapsules may be any kind of microcapsule disclosed herein or known in the art. The microcapsules may have a shell and a core material encapsulated by the shell. The core material of the microcapsules may include one or more fragrances. The shells of the microcapsules may be made from synthetic polymeric materials or naturally-occurring polymers. The microcapsules may be friable microcapsules. A friable microcapsule is configured to release its core material when its shell is ruptured. The rupture can be caused by forces applied to the shell during mechanical interactions. The microcapsules may have shells made from any material in any size, shape, and configuration known in the art. Some or all of the shells may include a polyacrylate material, such as a polyacrylate random copolymer. The microcapsules may also encapsulate one or more benefit agents. The benefit agent(s) include, but are not limited to, one or more of chromogens, dyes, cooling sensates, warming sensates, fragrances, oils, pigments, in any combination. When the benefit agent includes a fragrance, said fragrance may comprise from about 2% to about 80%, from about 20% to about 70%, from about 30% to about 60% of a perfume raw material with a C log P greater than −0.5, or even from about 0.5 to about 4.5. The microcapsules may encapsulate an oil soluble material in addition to the benefit agent. The microcapsule may be spray-dried to form spray-dried microcapsules. The personal care compositions may also include a parent fragrance and one or more encapsulated fragrances that may or may not differ from the parent fragrance. Some fragrances may be considered to be volatile and other fragrances may be considered to be or non-volatile. Further types and processes regarding microcapsules are disclosed in U.S. Pat. No. 9,687,425.

The composition may also contain one or more other delivery systems for providing one or more benefit agents, in addition or in place of the microcapsules. The additional delivery system(s) may differ in kind from the microcapsules. For example, wherein the microcapsule are friable and encapsulate a fragrance, the additional delivery system may be an additional fragrance delivery system, such as a moisture-triggered fragrance delivery system. Non-limiting examples of moisture-triggered fragrance delivery systems include cyclic oligosaccaride, starch (or other polysaccharide material), or combinations thereof. Further details regarding suitable starches and cyclic oligosaccharide are disclosed in U.S. Pat. No. 9,687,425.

The compositions may include one or more fragrances. As used herein, “fragrance” is used to indicate any odoriferous material. Any fragrance that is cosmetically acceptable may be used in the deodorant compositions. For example, the fragrance may be one that is a liquid at room temperature. Generally, the fragrance(s) may be present at a level from about 0.01% to about 40%, from about 0.1% to about 25%, from about 0.25% to about 20%, or from about 0.5% to about 15%, by weight of the personal care composition.

A wide variety of chemicals are known as fragrances, including aldehydes, ketones, and esters. More commonly, naturally occurring plant and animal oils and exudates comprising complex mixtures of various chemical components are known for use as fragrances. Non-limiting examples of the fragrances useful herein include pro-fragrances such as acetal pro-fragrances, ketal pro-fragrances, ester pro-fragrances, hydrolyzable inorganic-organic pro-fragrances, and mixtures thereof. The fragrances may be released from the pro-fragrances in a number of ways. For example, the fragrance may be released as a result of simple hydrolysis, or by a shift in an equilibrium reaction, or by a pH-change, or by enzymatic release. The fragrances herein may be relatively simple in their chemical make-up, comprising a single chemical, or may comprise highly sophisticated complex mixtures of natural and synthetic chemical components, all chosen to provide any desired odor. Suitable fragrances are also disclosed in U.S. Pat. Nos. 9,687,425, 4,145,184, 4,209,417, 4,515,705, and 4,152,272.

Cyclodextrin molecules are described in U.S. Pat. Nos. 5,714,137, and 5,942,217. Suitable levels of cyclodextrin are from about 0.1% to about 5%, alternatively from about 0.2% to about 4%, alternatively from about 0.3% to about 3%, alternatively from about 0.4% to about 2%, by weight of the composition.

### III. Method of Making

The deodorant composition can be made in any suitable manner known in the art, but generally follows the steps of 1) heating the emollient to a temperature of greater than 70° C., 2) adding the fatty acid salt gellant and heating until dissolved, 3) adding fragrance or any other labile material, 4) pouring the product into an appropriate container, and 5) allowing the product to cool and solidify.

### IV. Methods of Use and Methods of Reducing Body Malodor

The deodorant compositions of the present invention may be topically applied to the axilla or other area of the skin in any known or otherwise effective method for controlling malodor associated with perspiration. These methods comprise applying to the axilla or other area of the human skin an effective amount of the deodorant composition of the present invention, typically about 0.1 gram per axilla to about 2.0 gram per axilla. A method of use could be, for example, applying to a user a leave-on deodorant composition comprising from about 25% to about 70% of an ethoxylated or propoxylated fatty acid or an ethoxylated or propoxylated fatty alcohol, by weight of the composition, having a molecular weight of at least about 750 Daltons; and from about 2% to about 10% of a fatty acid salt gellant.

While some compositional components are listed in the methods section for illustration, the deodorant compositions in the methods can contain any combination of components as discussed above in the Deodorant Components section.

### V. Test Methods

### Hardness—Penetration Measurement for Deodorant Finished Products

The penetration test is a physical test method that provides a measure of the firmness of waxy solids and extremely thick creams and pastes with penetration values not greater than 250 when using a needle for D1321. The method is based on the American Society for Testing and Materials Methods D-5, D1321 and D217 and DIN 51 579 and is suitable for all solid antiperspirant and deodorant products.

A needle or polished cone of precisely specified dimensions and weight is mounted on the bottom of a vertical rod in the test apparatus. The sample is prepared as specified in the method and positioned under the rod. The apparatus is adjusted so that the point of the needle or cone is just touching the top surface of the sample. Consistent positioning of the rod is critical to the measured penetration value. The rod is then released and allowed to travel downward, driven only by the weight of the needle (or cone) and the rod. Penetration is the tenths of a millimeter travelled following release.

| APPARATUS | SUGGESTED TYPE (OR EQUIVALENT) |
|---|---|
| Penetrometer with Timer | Penetrometer Suitable For ASTM D-5 and D- |
| (see attached drawings | 1321 methods; Examples: Precision or |
| Attachment 1 and | Humboldt Universal Penetrometer (Humboldt |
| Attachment 2) | Manufacturing, Schiller Park, IL USA) or |
| | Penetrometer Model PNR10 or PNR12 |
| | (Petrolab USA or PetroTest GmbH). |
| Penetration Needles | GEL DEODORANTS: Needles as specified for |
| | ASTM Method D 1321/DIN 51 579, Officially |
| | certified, Taper-Tipped needle, No. H-1310, |
| | Humboldt Mfg. |

General Instructions—All Penetrometers Keep the instrument and needles/probes clean at all times, free from dust and grime. When not in use, store needles in a suitable container to avoid damage.
Periodic calibration should confirm:

Electronic Timer is correctly set. Verify against an independent stopwatch if unsure.

Shaft falls without visible signs of frictional resistance.

Ensure the total weight of the shaft and needle is 50±0.2 grams when the shaft is in free fall. Note: for modern, automated or digital systems this may be performed automatically and confirmed through annual calibration.

At time of use confirm:

Electronic Timer is correctly set to 5.0 seconds.

The appropriate needle is installed and is clean, straight and without obvious defects (visual inspection)

The penetrometer is level and the shaft is clean, straight and falls freely (visual inspection) Once level, avoid shifting the position of the unit to maintain level.

### Sample Preparation and Measurement

1. On a deodorant or antiperspirant stick that has cooled ambiently to a temperature between 22° C. and 26° C. for at least 24 hours, slice off top ½ inch of product to achieve a flat surface with a wire cutter drawn across the upper lip of the canister.
2. For the first sample to be tested, lubricate the needle by gently wiping with a lint-free tissue coated with a small amount of the product to be tested. This small amount is typically taken from the shaved top.
3. Place the canister in the appropriate location for the measurement. Locate the sample so the needle will penetrate the product 9-11 mm from the inside of the canister wall on the long axis.
4. Using the coarse and fine adjustments, align the height of the penetrometer mechanism head so that the point of the penetrating needle is just touching the surface of the sample.
A weak light at the side of the penetrometer which casts a shadow of the needle on the surface of the sample may be helpful in determining this contact. When a light area on the sample cannot be seen at the end of the tip of the needle's shadow, the needle height over the sample is correctly adjusted. The light should not be strong enough to heat or melt the sample surface. The needle should be just close enough to scratch the sample surface.
5. Perform the penetration measurement at this location by releasing the needle. Record the result.
6. Repeat Steps 2 through 4 at the other test point, i.e., at the other point 9-11 mm inside of the canister wall on the long axis.

To report results, units for penetration are tenths of a millimeter ( 1/10 mm=100 microns). Report the average results of at least 4 total measurements from 2 different sticks. Report the average result of the measurements to the nearest tenth of a millimeter.

### Examples

| Material | A | B | C | D | E | F |
|---|---|---|---|---|---|---|
| PP-11 stearyl ether | | | 35 | | 20 | 20 |
| PPG-15 stearyl ether | 37 | 36 | | 36 | 18 | 18 |
| PPG-15 butyl ether | | | | 28 | | 29.4 |
| cyclopentasiloxane | 25.8 | 26.8 | 29.3 | | 24.4 | |
| Dipropylene glycol | 15 | 15 | 15 | 15.3 | 15 | 15 |
| Sodium Stearate | 5 | 4.5 | 5 | 5 | 5 | 4.5 |
| C12-15 alkyl benzoate | 5 | 5 | 5 | 5 | 4.5 | 2 |
| water | 5 | 4.5 | 4.5 | 4.5 | 5 | 5 |
| Polyglycerol 3 laurate | 4 | 3 | 3 | | 3 | |
| Mineral oil | 0.1 | 0.1 | 0.1 | 0.1 | | |
| 1,2 Hexanediol | 1 | | | | 2 | |
| Hexanediol and capryl | | 3 | | 3 | | 3 |
| glycol | | | | | | |
| fragrance | 2 | 2 | 3 | 3 | 3 | 3 |
| hexamidine diisethionate | 0.05 | 0.05 | 0.05 | 0.05 | 0.05 | 0.05 |
| BHT preservative | 0.05 | 0.05 | 0.05 | 0.05 | 0.05 | 0.05 |
| | 100 | 100 | 100 | 100 | 100 | 100 |

Examples A-F can be made by any appropriate method known in the art for making deodorant solid sticks. It is often convenient to mix the water soluble ingredients first, including the sodium stearate, then heat to 75° C. to begin dissolution of the stearate. Next, the water insoluble materials can be added in any desired order, and the temperature increased to 85° C. and held until a clear solution is observed. The solution is then cooled to 70° C. to add fragrance, and then poured into an appropriate deodorant stick package.

Throughout this specification, components referred to in the singular are to be understood as referring to both a single or plural of such component.

All percentages stated herein are by weight unless otherwise specified.

Every numerical range given throughout this specification will include every narrower numerical range that falls within such broader numerical range, as if such narrower numerical range were all expressly written herein. For example, a stated range of “1 to 10” should be considered to include any and all subranges between (and inclusive of) the minimum value of 1 and the maximum value of 10; that is, all subranges beginning with a minimum value of 1 or more and ending with a maximum value of 10 or less, e.g., 1 to 6.1, 3.5 to 7.8, 5.5 to 10, etc.

Further, the dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A deodorant composition comprising:
a. from about 25% to about 70%, by weight of the composition, of an emollient selected from the group consisting of a propoxylated fatty alcohol, a propoxylated fatty acid, an ethoxylated propoxylated fatty alcohol, an ethoxylate propoxylated fatty acid and combinations thereof, each emollient having a molecular weight of at least about 750 Daltons; and
b. from about 2% to about 10% of a fatty acid salt gellant.

2. The deodorant composition of claim 1, further comprising at most about 30% of a polyhydric alcohol.

3. The deodorant composition of claim 1, further comprising from about 2% to about 8% water, by weight of the composition.

4. The deodorant composition of claim 1, further comprising a fragrance.

5. The deodorant composition of claim 1, further comprising an antibacterial.

6. The deodorant composition of claim 1, wherein the deodorant composition is translucent.

7. The deodorant composition of claim 1, wherein the deodorant composition is clear.

8. The deodorant composition of claim 1, wherein the deodorant composition has a penetration value of at most 120 units.

9. The deodorant composition of claim 1, wherein the deodorant composition has a penetration value from about 80 units to about 120 units.

10. The deodorant composition of claim 1, wherein any emollient having a molecular weight of at least about 750 Daltons is water-dispersible.

11. The deodorant composition of claim 1, wherein the composition is in the form of a solid deodorant.

12. The deodorant composition of claim 1, wherein the composition is substantially free of cyclopentasiloxane.

13. The deodorant composition of claim 1, comprising from about 25% to about 50%, by weight of the composition, of emollients having a molecular weight of at least about 750 Daltons.

14. The deodorant composition of claim 1, wherein any emollient having a molecular weight of at least about 750 Daltons is liquid.

15. A deodorant composition comprising:
a. from about 25% to about 70%, by weight of the composition, of an emollient selected from the group consisting of a propoxylated fatty alcohol, a propoxylated fatty acid, an ethoxylated propoxylated fatty alcohol, an ethoxylate propoxylated fatty acid and combinations thereof, each emollient having a molecular weight of at least about 750 Daltons; and
b. at most about 30%, by weight of the composition, of a polyhydric alcohol.

16. The deodorant composition of claim 15, further comprising from about 2% to about 8% water.

17. The deodorant composition of claim 15, further comprising an antimicrobial.

18. A deodorant composition comprising:
a. from about 25% to about 70%, by weight of the composition, of an emollient selected from the group consisting of a propoxylated fatty alcohol, a propoxylated fatty acid, an ethoxylated propoxylated fatty alcohol, an ethoxylate propoxylated fatty acid and combinations thereof, each emollient having a molecular weight of at least about 750 Daltons; and
b. from about 2% to about 8% water.

19. The deodorant composition of claim 18, wherein the composition is free of aluminum and free of cyclopentasiloxane.

20. A deodorant composition comprising:
a. from about 25% to about 70%, by weight of the composition, of a liquid emollient selected from the group consisting of a propoxylated fatty alcohol, a propoxylated fatty acid, an ethoxylated propoxylated fatty alcohol, an ethoxylate propoxylated fatty acid and combinations thereof; and
b. from about 2% to about 10% of a fatty acid salt gellant.
